(19) United States
(12) Patent Application Publication
(10) Pub. No.: US 2022/0193200 A1
(43) Pub. Date: Jun. 23, 2022

(54) TANNIC ACID/FE (III) NANOPARTICLES AND METHODS OF DRUG DELIVERY

(71) Applicants: The Johns Hopkins University, Baltimore, MD (US); Sun Yat-Sen University, Guangzhou (CN)

(72) Inventors: Hai-Quan Mao, Baltimore, MD (US); Zhiyu He, Baltimore, MD (US); Yizong Hu, Baltimore, MD (US); Yongming Chen, Guangzhou (CN); Lixin Liu, Guangzhou (CN)

(21) Appl. No.: 17/441,917

(22) PCT Filed: Mar. 22, 2020
(86) PCT No.: PCT/US2020/023876

§ 371 Date: Sep. 22, 2021

Related U.S. Application Data
(60) Provisional application No. 62/822,085, filed on Mar. 22, 2019.

(30) Foreign Application Priority Data
Mar. 22, 2019 (CN) 201910222916.4

(51) Int. Cl. A61K 38/26; A61K 9/16

(52) U.S. Cl. CPC A61K 38/26; A61K 9/1694; A61K 9/1605; A61K 9/1617

(57) ABSTRACT

Nanoparticle are described comprising a tannic acid, a trivalent metal ion, and a pharmaceutical agent comprising a water-soluble biologically active agent, such as a protein, a water-soluble peptide, small molecule or a combination thereof and methods of making and using the nanoparticles. Some nanoparticles of the present invention include 30-60% (w/w) of a tannic acid, 0.1-20% (w/w) of a trivalent metal ion, and 1-50% (w/w) of a pharmaceutical agent.

### REFERENCE TO RELATED APPLICATIONS

This application claims the benefit of U.S. Provisional Patent Application No. 62/822,085, filed on Mar. 22, 2019, and Chinese Patent Application 201910222916.4, filed Mar. 22, 2019, the content of each of the aforementioned applications is herein incorporated by reference in their entirety.

### INCORPORATION-BY-REFERENCE OF MATERIAL SUBMITTED ELECTRONICALLY

The instant application contains a Sequence Listing that has been submitted in ASCII format via EFS-Web and is hereby incorporated by reference in its entirety. Said ASCII copy, created on Mar. 2, 2020, is named P15629-02_ST25.txt and is 1,545 bytes in size.

### BACKGROUND OF THE INVENTION

Type 2 diabetes (T2D), which contributes 90% of all diabetic cases, poses a significant public health burden all over the world [1]. One of the most effective drugs for T2D treatment, glucagon-like peptide-1 (GLP-1) [2], has major challenges due to its short half-life in blood (less than 2 min) as a result of fast clearance by proteolytic enzyme dipeptidyl peptidase-4 (DDP-4) [3] that greatly hinder its wider clinical applications. There have been many efforts in recent years to generate analogs of GLP-1 with improved circulation time [4-6]. John et al. isolated a 39 amino acid peptide exendin-4 in the salivary of Gila monster [7] that has a 20- to 30-times longer half-life in the blood than GLP-1, and shares 53% sequence homology and many glucoregulatory actions, including glucose-dependent enhancement of insulin secretion, glucose-dependent suppression of inappropriately high glucagon secretion, slowing of gastric emptying and reduction of food intake [8]. In addition, exendin-4 has been shown to promote 0-cell proliferation and islet neogenesis from precursor cells both in vitro and in vivo [9]. However, the poor serum stability of the peptide requires it to be injected twice every day, leading to poor patient compliance.

Various strategies have been developed to enhance the durability and effectiveness of exendin-4 treatment [10-17]. Chemical modifications such as PEGylation [18] and conjugation to albumin [19] could increase their serum stability and circulation time, but these chemical structural modifications may decrease the drug bioactivity. The other strategy aimed at a sustained release of the drug by packaging exendin-4 into biomaterials such as poly (lactic acid-co-glycolic acid) (PLGA) microspheres. However, the wide particle size distribution, limited colloidal stability, and poor loading capability (˜5%) resulted in suboptimal release profiles [11]. The formulation in both cases suffers from laborious processing methods, low throughput, and poor reproducibility, which will eventually hinder their translation.

Various strategies have been developed to enhance the durability and effectiveness of GLP-1 receptor agonist peptide treatment, using exendin-4 for example [10-17]. Chemical modifications such as PEGylation [18] and conjugation to albumin [19] could increase their serum stability and circulation time, but these chemical structural modifications may decrease the drug bioactivity. The other strategy aimed at a sustained release of the drug by packaging exendin-4 into biomaterials such as poly(lactic acid-co-glycolic acid) (PLGA) microspheres. However, the wide particle size distribution, limited colloidal stability, and poor loading capability (˜5%) resulted in suboptimal release profiles [11]. The formulation in both cases suffers from laborsome processing, low throughput, and poor reproducibility, which will eventually hinder their translation.

### SUMMARY OF THE INVENTION

In accordance with an embodiment, the present invention provides a nanoparticle comprising a tannic acid, a trivalent metal ion, and a pharmaceutical agent comprising a water-soluble protein, a water-soluble peptide, or a combination thereof.

In accordance with another embodiment, the present invention provides a nanoparticle comprising a tannic acid, a trivalent metal ion, and a pharmaceutical agent comprising a GLP-1 receptor agonist, or a combination thereof.

In accordance with a further embodiment, the present invention provides a method for treating a subject with Type 2 Diabetes comprising administering to the subject an effective amount of a composition comprising a tannic acid, a trivalent metal ion, and a pharmaceutical agent comprising a GLP-1 receptor agonist, or a combination thereof.

In accordance with an embodiment, the present invention provides methods for making a nanoparticle comprising a tannic acid, a trivalent metal ion, and a pharmaceutical agent comprising a water-soluble protein, a water-soluble peptide, or a combination thereof comprising the use of flash nanocomplexation of the components of said nanoparticle.

### BRIEF DESCRIPTION OF THE DRAWINGS

FIGS. 1A-1B. Schematic illustrations of (1A) FNC preparation of TA/exendin-4/Fe³⁺ nanoparticles and crosslinking structures, and (1B) pH-dependent payload release as a result of dissociation of crosslinks.

FIGS. 2A-2C. Nanoparticle assembly of TA/exendin-4/Fe³⁺ complexes. (2A) Effect of volumetric flow rate of FNC on nanoparticle size distribution of NP-4 formulation. Curves (filled circles) and bars represent mean±S.D. (n=3) of measurements from three different preparations; (2B) TEM images of exendin-4-loaded NP-4 prepared at a flow rate of 0.5 or 20 mL/min. (1) Magnified view of nanoparticles in red circles; (2) Magnified view of aggregates; (2C) FTIR spectra of TA, exendin-4, FeCl₃, TA/Fe³⁺ complexes, TA/exendin-4 complexes and TA/exendin-4/Fe³⁺ nanoparticles.

FIGS. 3A-3D. Characterizations of exendin-4 loaded nanoparticles. 3A) Effect of TA concentration in FNC preparation on the nanoparticle size and encapsulation efficiency of exendin-4; 3B) Colloidal stability as reflected by the average size of the exendin-4-loaded nanoparticles prepared by different TA concentrations in FNC; 3C) In vitro release profile of exendin-4 from nanoparticles prepared by different TA concentrations in DI water (pH 5.0) and 10 mM PBS (pH 7.4); 3D) In vitro release profile of exendin-4 from NP-4 in PBS (pH 7.4) at different concentrations (10, 20 and 30 mM). All data are shown as mean±S.D. (n=4).

FIGS. 4A-4C. Sustained release of exendin-4 from nanoparticles in Balb/c mice following i.p. injection. (4A) In vivo fluorescence imaging of mice treated with i.p. single dose of Cy 7.5-labeled free exendin-4 (dose=500 μg/kg) and exendin-4-loaded nanoparticles (dose=500 μg/kg) at different time points; (4B) Fluorescence intensity (mean±S.D.) of the Cy 7.5-labeled free exendin-4 and exendin-4-loaded nanoparticles in peritoneal cavity given by IVIS images (n=3); (4C) Fluorescence imaging of blood samples collected from mice dosed with Cy 7.5-labeled free exendin-4 or nanoparticles at different time points.

FIGS. 5A-5G. In vivo efficacy of exendin-4-loaded nanoparticles in db/db mice (a T2D model). (5A) Blood glucose levels of db/db mice after single i.p. dosing of free exendin-4 (3 mg/kg) and exendin-4-loaded NP-4 (equivalent to 3 or 6 mg/kg exendin-4). Control groups were PBS-treated db/db T2D model mice and db/m healthy mice; (5B) Area under curve (AUC) of the blood glucose level versus time from 0 to 204 h. ***p<0.001 when comparing with the free exendin-4 group. ^{###}p<0.001 when comparing with the NP-4 (3 mg/kg) group; (5C) Body weights of mice from different groups, monitored for 8 days after treatment; (5D) Oral glucose tolerance test (OGTT) of db/db mice after single i.p. dosing of free exendin-4 and NP-4. Glucose was given through oral gavage at 1 h after treatment; (5E) AUC of the blood glucose level versus time in response to OGTT. ^{ΔΔΔ}p<0.001 when comparing with the PBS control; (5F) Exendin-4 concentration detected in the blood as a function of time following treatment with free exendin-4 or NP-4; (5G) AUC of blood exendin-4 concentration vs. time for the two treatment groups. ***p<0.001 when comparing with the free exendin-4 group. Data is shown as mean±S.D. (n=5).

FIGS. 6A-6C. Pathological improvement in T2D mice after treatment of exendin-4-loaded nanoparticles. (6A) Tissue morphology in major organs upon dosage of free exendin-4 or NP-4 (3 mg/kg), as assessed by H&E staining for tissue samples collected on Day 9 after treatment; Scale bar=100 m. (6B) Enzyme activities of alkaline phosphatase (ALP), aspartate transaminase (AST), alanine aminotransferase (ALT), γ-glutamyl transpeptidase (γ-GT) in serum samples collected on Day 9 after treatment; (6C) Levels of blood urea nitrogen (BUN) and creatinine (CR) in serum samples collected on Day 9 after treatment. Data is shown as mean±S.D. (n=5).

FIGS. 7A-7C. Characterization of blank tannic acid/extendin-4 complex. (7A) Dynamic light scattering (DLS) analysis of tannic acid/extendin-4 complex immediately after preparation; (7B) DLS analysis of tannic acid/extendin-4 complex at 15 min post-preparation; (7C) Images of precipitated of tannic acid/extendin-4 complex at 30 min post-preparation (1) and the pellet of tannic acid/extendin-4 complex post centrifugation (2).

FIGS. 8A-8B. (8A) Image of Tyndall scattering of TA/Al³⁺ complexes and TA/Zn²+ complexes; (8B) DLS analysis of TA/exendin-4/Zn²⁺ complexes.

FIG. 9. Nanoparticle size distribution of NP-4 under a flow rate of 25 mL/min.

FIG. 10. Size distribution of Cy 7.5 labeled exendin-4 nanoparticles formulated with a TA concentration of 3 mg/mL (pH 5.0), exendin-4 concentration of 1 mg/mL (pH 7.4) and Fe³⁺ concentration of 0.05 mg/mL (pH 2.0), and FNC flow rate of 20 mL/min.

FIG. 11. Cell viability results (MTT assay) with different exendin-4 doses of NP-4. Data is shown as mean±S.D. (n=6).

FIG. 12. Blood glucose levels (BGL) of db/db mice after i.p. single dose of free exendin-4, NP-4 or NP-5, all given at 3 mg/kg exendin-4 dose level. Control groups including PBS-treated db/db T2D model mice, NP-4 (3 mg/kg) treatment, and db/m healthy mice were included here for comparison. Data is shown as mean±S.D. (n=5).

### DETAILED DESCRIPTION OF THE INVENTION

The present inventors have now designed a tannic acid (TA)/exendin-4/Fe³⁺ ternary nanoparticle system where molecules or peptides, such as exendin-4, binds to TA through hydrogen bonding and hydrophobic interactions, wherein the nanocomplexes formed are stabilized by ionic chelation of classic phenol-iron (III) coordination reaction between TA and Fe³⁺ ions [20-22]. The inventors hypothesized that a strong and multivalent TA-protein interaction [23-25] would render a high complexation affinity and stability, and enable a prolonged release of the complexed peptides or other molecules, because of strong complex association. To achieve efficient control over the reaction kinetics to allow uniform assembly among the three components, the inventors use a flash nanocomplexation (FNC) technique [26, 27] to mix the TA, molecule, and FeCl₃ solutions via turbulent mixing in a confined impinging jet (CIJ) microchamber, where nanoparticles were formed within tens of milliseconds [28]. The inventors demonstrated proof of concept of the controlled assembly by creating TA/exendin-4/Fe³⁺ ternary nanoparticles. The inventors then examined the effect of FNC process parameters on nanoparticle characteristics and the release profiles of exendin-4. Using an optimized nanoparticle formulation, the inventors assessed the effectiveness of these ternary nanoparticles in regulating blood glucose concentration in a T2D mouse model.

Therefore, in accordance with an embodiment, the present invention provides a nanoparticle comprising a tannic acid, a trivalent metal ion, and a pharmaceutical agent comprising a water-soluble protein, a water-soluble peptide, or a combination thereof.

In some embodiments, the nanoparticles of the present invention typically have a mass ratio of the tannic acid to the trivalent metal ion in the range between 10 and 100; 15 and 95; 20 and 90; 25 and 85; 30 and 80; 35 and 75; or 40 and 70. Some nanoparticle of the present invention comprise 30-60% or 40-50% (w/w) of the tannic acid. Some nanoparticle of the present invention comprise 0.1-20%, 1-15%, or 2-10% (w/w) of the trivalent metal ion. Some nanoparticles of the present invention comprise 1-50%, 5-45%, 10-40%, or 15-35% (w/w) of the pharmaceutical agent.

As used herein, the term “trivalent metal ion” means a metal atom with a +3 valence. Typical trivalent ions are Fe (III), Al (III), Cr (III), but could also include Ce (III), Eu (III), Tb (III), Er (III), Nd (III), and Gd (III). Examples of suitable trivalent metal ion used in the present invention include Fe (III), Al (III) or a combination thereof.

Suitable peptides used in the present invention may comprise 5 to 150, 5 to 100, or 5 to 50 amino acid residues. Suitable proteins used in the present invention have a size in the range from 5 to 200 kDa, 5 to 150 kDa, or 5 to 100 kDa. Examples of suitable water-soluble peptide or water-soluble proteins used in the present invention include antibodies; antibody fragments; hormones; hormone receptors; receptor ligands; cytokines; growth factors; extendin-4 or a combination thereof. Some nanoparticle of the present invention include extendin-4, 65.6% (w/w) of the tannic acid, 32.8% (w/w) of the extendin-4, and 1.6% (w/w) of a trivalent metal ion consisting of Fe (III). Other nanoparticles of the present invention include 79.2% (w/w) of the tannic acid, 19.8% (w/w) of the extendin-4, and 1.0% (w/w) of the trivalent metal ion consisting of Fe (III).

Another embodiment of the present invention is a method of drug delivery. A nanoparticle of the present invention is administered to a subject. The nanoparticle adheres to cells of the subject and delivers a pharmaceutical agent to the cells. Nanoparticles of the present invention maybe administered by oral delivery, intravenous injection, intramuscular injection, intraperitoneal injection, subcutaneous injection, or a combination thereof.

Another embodiment of the present invention is a flash nanocomplexation (FNC) method of continuously generating uniform tannic acid/peptide or protein/trivalent metal ions nanoparticles. The method includes flowing a first stream comprising tannic acid dissolved in water having a concentration of the tannic acid in the range of 0.2 to 40 mg/mL, 1.0 to 30 mg/mL, 2.0 or 20 mg/mL at a first variable flow rate into a confined chamber.

Flowing a second stream comprising one or more of a water-soluble peptide or water-soluble protein having a concentration in the range of 0.1 to 20 mg/mL, 1.0 to 15 mg/mL, or 1.5 to 10 mg/mL at a second variable flow rate into the confined chamber.

Flowing a third stream comprising one or more of a water-soluble trivalent metal ion having a concentration in the range of more 0.005 to 1 mg/mL, 0.05 to 0.9 mg/mL, or 0.5 to 0.8 mg/mL at a third variable flow rate into the confined chamber.

Impinging the first stream, the second stream and the third stream in the confined chamber, thereby causing the tannic acid, the one or more water-soluble trivalent metal ions and the one or more water-soluble peptide or water-soluble protein to undergo a complexation process that continuously generates tannic acid/peptide or protein/metal ions (III) ternary nanoparticles.

A suitable confined chamber includes a 3-inlet confined impingement jet mixer device. Suitable first, second, and third variable flow rates are each in the range from 0.5 to 100 mL/min, 1.0 to 90 mL/min, 5.0 to 80 mL/min, 10 to 70 mL/min, or 20 to 70 mL/min. As discussed above, water-soluble trivalent metal ions used in the present invention include Fe (III), Al (III), or a combination thereof.

A suitable tannic acid concentration used in the present invention is in the range from 0.5 to 10 mg/ml, 1 to 9 mg/ml, 2 to 8 mg/ml, 3 to 7 mg/ml, or 4 to 6 mg/ml and the first stream may have a pH in the range of 3.0 to 7.0 or 4.0 to 6.0.

A suitable water-soluble peptide or protein concentration used in the present invention is in the range from 0.5 to 5 mg/ml, 1 to 4 mg/ml, or 2 to 3 mg/ml and the second stream may have a pH in the range of 5.5 to 8.0 or 6.5 to 7.5. A suitable water-soluble trivalent metal ion concentration is in the range from 0.005 to 0.5 mg/ml, 0.05 to 0.4 mg/ml, 0.5 to 0.3 mg/ml, or 0.1 to 0.2 mg/ml and the third stream has a pH in the range of 1.0 to 4.0 or 2.0 to 3.0.

Suitable tannic acid/peptide or protein/metal ions (III) ternary nanoparticles generated by the methods of the present invention have a size in the range from about 20 nm to about 500 nm in diameter. Suitable tannic acid/peptide or protein/metal ions (III) ternary nanoparticles generated by the methods of the present invention have a polydispersity index in the range from about 0.02 to about 0.4 or about 0.5 to about 02.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “activity” refers to the ability of a pharmaceutical agent to perform its function after being transported by the tannic acid/FE(III) nanoparticles of the present invention.

The term “antibody,” as used in this disclosure, refers to an immunoglobulin or a fragment or a derivative thereof, and encompasses any polypeptide comprising an antigen-binding site, regardless of whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)₂, Fv, scFv, Fd, dAb, and other antibody fragments that retain antigen-binding function, i.e., the ability to bind, for example, PD-L1, specifically. Typically, such fragments would comprise an antigen-binding domain.

The terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as “epitope” or “antigenic determinant.” An antigen-binding domain typically comprises an antibody light chain variable region (V_{L}) and an antibody heavy chain variable region (V_{H}), however, it does not necessarily have to comprise both. For example, a so-called Fd antibody fragment consists only of a V_{H} domain, but still retains some antigen-binding function of the intact antibody.

Binding fragments of an antibody are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in a F(ab′)2 fragment in which the two arms of the antibody molecule remain linked and comprise two-antigen binding sites. The F(ab′)2 fragment has the ability to crosslink antigen. “Fv” when used herein refers to the minimum fragment of an antibody that retains both antigen-recognition and antigen-binding sites. “Fab” when used herein refers to a fragment of an antibody that comprises the constant domain of the light chain and the CHI domain of the heavy chain.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “disease” is meant any condition or disorder that damages or interferes with the normal function of a cell, tissue, or organ. Examples of diseases include diabetes or cancer.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “peptide agonist” is meant glucagon-like peptide (GLP) receptor agonist.

By “glucagon-like peptide (GLP) receptor agonist” is meant a peptide that promotes insulin secretion, like Exendin-4 (HGEGTFTSDLSKQMEEEAVRLFIEWLKNGGPSSGAPPPS) (SEQ ID NO: 1), Liraglutide (HAEGTFTSDVSSYLEGQAAKEFIAWLVRGRG) (SEQ ID NO: 2), and Lixisenatide (HGEGTFTSDLSKQMEEEAVRLFIEWLKNGGPSSGAPPSKKKKKK) (SEQ ID NO: 3), and functional fragments and portions thereof.

By “nanoparticle” is meant a particle having the size in the range of 10 nm to 1000 nm, 10 nm to 500 nm, 10 nm to 200 nm, or 10 to 100 nm, as examples.

By “obtaining” as in “obtaining an agent” is meant synthesizing, purchasing, or otherwise acquiring the agent.

By “mAb” is meant monoclonal antibody. Antibodies of the invention comprise without limitation whole native antibodies, bispecific antibodies; chimeric antibodies; Fab, Fab′, single chain V region fragments (scFv), fusion polypeptides, and unconventional antibodies.

By “polypeptide,” “peptide” and “protein” is meant terms used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

A “reference” refers to a standard or control conditions such as a sample (human cells) or a subject that is a free, or substantially free, of an agent or a nanoparticle of the present invention including an agent.

As used herein, the term “subject” is intended to refer to any individual or patient to which the method described herein is performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

### Pharmaceutical Applications

Embodiments of the disclosure concern methods and/or compositions for delivering pharmaceutical agents to a subject to treat or prevent disease. In certain, individuals with cardiovascular disease, cancer, or diabetes may be treated with nanoparticles of the present invention including one or more pharmaceutical agent(s). Suitable pharmaceutical agents that may be delivered by the nanoparticles of the present invention include proteins, peptides, nucleic acids, and chemicals including antibodies, RNA, DNA, and small chemical entities as examples. Known pharmaceutical agents delivered by the nanoparticles of the present invention include Exendin-4, Liraglutide, Insulin, Teduglutide, Lixisenatide, Abaloparatide, and a combination thereof, as examples.

Non-limiting examples of biologically active agents include following: anabolic agents, androgenic steroids, anti-angiogenic compounds, anti-cancer compounds, anti-allergenic materials, anti-cholesterolemic and anti-lipid agents, anti-coagulants, anti-convulsants, anti-hypertensive agents, anti-infective agents, anti-inflammatory agents such as steroids, non-steroidal anti-inflammatory agents, anti-malarials, anti-nauseants, anti-neoplastic agents, anti-pyretic and analgesic agents, anti-spasmodic agents, anti-thrombotic agents, biologicals, cardioactive agents, cerebral dilators, coronary dilators, decongestants, diuretics, diagnostic agents, erythropoietic agents, mitotics, mucolytic agents, growth factors, neuromuscular drugs, nutritional substances, peripheral vasodilators, progestational agents, prostaglandins, vitamins, and prodrugs.

Still further, the following listing of peptides, proteins, and other large molecules may also be used, such as interleukins 1 through 18, including mutants and analogues; interferons a, y, and which may be useful for cartilage regeneration, hormone releasing hormone (LHRH) and analogues, gonadotropin releasing hormone transforming growth factor (TGF); fibroblast growth factor (FGF); tumor necrosis factor-α); nerve growth factor (NGF); growth hormone releasing factor (GHRF), epidermal growth factor (EGF), connective tissue activated osteogenic factors, fibroblast growth factor homologous factor (FGFHF); hepatocyte growth factor (HGF); insulin growth factor (IGF); invasion inhibiting factor-2 (IIF-2); bone morphogenetic proteins 1-7 (BMP 1-7); somatostatin; thymosin-a-y-globulin; superoxide dismutase (SOD); and complement factors, and biologically active analogs, fragments, and derivatives of such factors, for example, growth factors.

An active agent and a biologically active agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se, as well as pharmaceutically acceptable, pharmacologically active salts, esters, amides, prodrugs, metabolites, analogs etc.

In certain embodiments, pharmaceutical agents increasing or decreasing expression or activity of one or more proteins may be delivered by nanoparticles of the present invention. These nanoparticles are provided in an amount and duration enough to ameliorate at least one symptom of a specific disease. The level of expression may increase (or decrease depending upon the pharmaceutical agent administered to a subject) by at least 2, 3, 4, 5, 10, 25, 50, 100, 1000, or more fold expression compared to the level of expression in a standard, in at least some cases.

### Pharmaceutical Preparations

Pharmaceutical compositions of the present invention comprise an effective amount of one or more of the nanoparticles of the present invention including a pharmaceutical agent, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition including nanoparticles of the present invention comprising at least one additional active ingredient, or pharmaceutical agent, will be known to those of skill in the art in light of the present disclosure, as exemplified by Remington: The Science and Practice of Pharmacy, 21^{st} Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The nanoparticles of the present invention including a pharmaceutical agent may comprise different types of carriers depending on whether it is to be administered in solid, liquid or aerosol form, and whether it need to be sterile for such routes of administration as injection. The present compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The pharmaceutical agent that is part of the nanoparticle of the present invention may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the nanoparticles of the present invention including a pharmaceutical agent may be suitable for administration in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the nanoparticles of the present invention including a pharmaceutical agent are combined with the carrier in any convenient and practical manner, i.e., by solution, suspension, emulsification, admixture, encapsulation, absorption and the like. Such procedures are routine for those skilled in the art. In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

The actual dosage amount of nanoparticles of the present invention including a pharmaceutical agent administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, nanoparticles of the present invention including a pharmaceutical agent may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Alimentary Compositions and Formulations

In one embodiment of the present disclosure, the nanoparticles of the present invention including a pharmaceutical agent are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these nanoparticles of the present invention including a pharmaceutical agent may be formulated with an inert diluent or with an assimilable edible carrier, or they may be enclosed in hard- or soft-shell gelatin capsule, or they may be compressed into tablets, or they may be incorporated directly with the food of the diet. In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety).

The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively, the compositions may be fashioned into a tablet or solution form that may be placed under the tongue or otherwise dissolved in the mouth. Additional formulations, which are suitable for other modes of alimentary administration, include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

### Parenteral Compositions and Formulations

In further embodiments, nanoparticles of the present invention including a pharmaceutical agent may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety). Solutions of the active compounds, in nanoparticles of the present invention, as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

### Miscellaneous Pharmaceutical Compositions and Formulations

In other preferred embodiments of the invention, the nanoparticles of the present invention including a pharmaceutical agent may be formulated for administration via various miscellaneous routes, for example, topical (i.e., transdermal) administration, mucosal administration (intranasal, vaginal, etc.) and/or inhalation. Pharmaceutical compositions for topical administration may include the active compound formulated for a medicated application such as an ointment, paste, cream or powder. Ointments include all oleaginous, adsorption, emulsion and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety). The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### Kits of the Disclosure

Any of the compositions described herein may be comprised in a kit. In a non-limiting example, nanoparticles of the present invention including a pharmaceutical agent may be comprised in a kit. The kits may comprise a suitable nanoparticle of the present invention including a pharmaceutical agent and, in some cases, one or more additional agents. The component(s) of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the nanoparticles of the present invention including a pharmaceutical agent and any other reagent containers in close confinement for commercial sale. Such containers may include injection or blow-molded plastic containers into which the desired vials are retained. When the components of the kit are provided in one and/or more liquid solutions, the liquid solution is an aqueous solution, with a sterile aqueous solution being particularly preferred. The nanoparticles of the present invention including a pharmaceutical agent may be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit. However, the components of the kit may be provided as dried powder(s). When reagents and/or components are provided as a dry powder, the powder can be reconstituted by the addition of a suitable solvent. It is envisioned that the solvent may also be provided in another container means.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation

Materials

Tannic acid and ferric trichloride (FeCl₃) were purchased from Sigma Aldrich. Exendin-4 (HGEGTFTSDLSKQMEEEAVRLFIEWLKNGGPSSGAPPPS) (SEQ ID NO: 1) was purchased from Taiye Biopharmaceuticals Co. Ltd (China). Cyanine 7.5 (Cy 7.5) NHS ester was purchased from Lumi-Probe (USA). Assay kits for alkaline phosphatase (ALP), aspartate transaminase (AST), alanine aminotransferase (ALT), γ-glutamyl transpeptidase (γ-GT), blood urea nitrogen (BUN) and creatinine (CR) were purchased from Jiancheng Biotech Co. Ltd. (China). Vivaspin® 500 ultrafiltration tubes were purchased from Sartorius (USA). Float-A-Lyzer® G2 Dialysis Device was purchased from Spectrum Labs (USA). NanoOrange Protein Quantitation Kit was purchased from Thermo Fisher Scientific (USA). Exendin-4 ELISA kit was purchased from Phoenix Biotech (USA).

Flash Nanocomplexation (FNC) Platform Set-Up and Optimization for Exendin-4-Loaded Nanoparticles

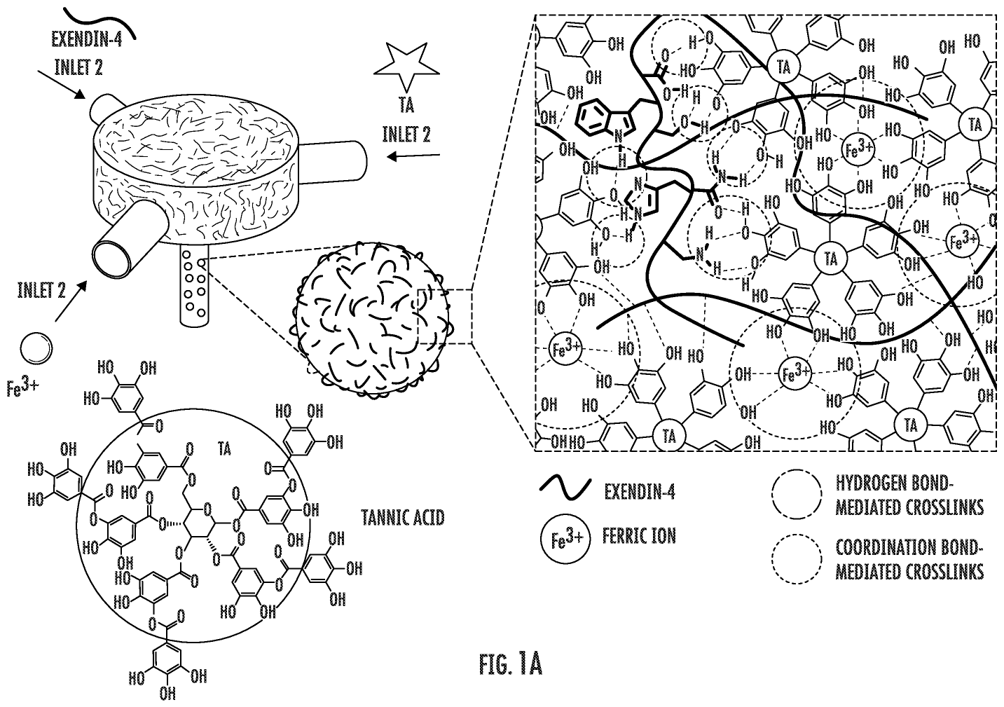

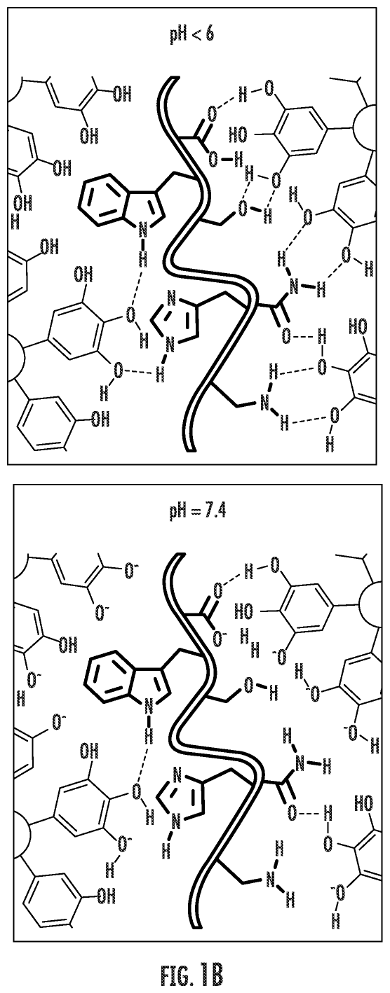

Three sets of working solutions were prepared: (1) TA was dissolved in 50 mM HEPES (pH 5.0) to generate 5 solutions with concentrations of 1, 1.5, 2, 3, and 4 mg/mL, respectively; (2) Exendin-4 was dissolved in deionized (DI) water at a concentration of 1 mg/mL and the pH was adjusted to 7.4 with 10 mM NaOH solution; (3) 0.05 mg/mL FeCl₃ solution was prepared by dilution from a 0.5 mg/mL solution prepared in concentrated HCl solution to avoid hydrolysis, and the final pH of the solution was adjusted to 2.0. The TA, exendin-4, and FeCl₃ solutions were introduced into each inlet of a 3-inlet CIJ mixer device (FIG. 1) with the flow rate of each stream kept the same and controlled by a digital syringe pump (New ERA, NE-4000, USA). The first one mL mixture solution was discarded in each production to account for the time it takes to establish homogenous mixing condition. The pH of the final nanoparticle suspension was pH 4.7. In preparation of control particles from TA and exendin-4 complexes, the FeCl₃ stream was replaced by a 10 mM HCl solution. For optimization of the encapsulation efficiency of exendin-4, the TA/exendin-4/Fe³⁺ mass ratio was tuned by TA concentration while keeping all the other parameters constant (exendin-4: 1 mg/mL, pH 7.4; and FeCl₃: 0.05 mg/mL, pH 2.0). Nanoparticles prepared by TA solution with a concentration of 1, 1.5, 2, 3 and 4 mg/mL were termed NP-1, NP-2, NP-3, NP-4, and NP-5, respectively.

Nanoparticle Characterizations

Dynamic light scattering (DLS) (for size and polydispersity index measurements) and phase analysis light scattering (PALS) (for zeta potential measurements) of the nanoparticles were conducted on a Malvern Zetasizer Nano ZS at room temperature. The morphology of the nanoparticles was assessed by transmission electron microscopy (TEM) on FEI Tecnai 12 (USA). The encapsulation efficiency (EE) of exendin-4 was assessed by measuring unencapsulated exendin-4 in the supernatant. Briefly, the nanoparticle suspension was filtered using an ultrafiltration tube (MWCO 300 kDa) at 300×g for 20 min at room temperature. The concentrations of exendin-4 in the filtrate and the starting exendin-4 solution used for nanoparticle assembly were measured using NanoOrange® Protein Quantitation Kit using a standard curve generated with a series of exendin-4 solutions with known concentrations. EE was calculated using Equation (1):

$$EE ( \% ) = ( 1 - \frac{Amount of unecapsulated peptide}{Total amount of peptide added} ) \times 100 \% \qquad ( 1 )$$

FT-IR Analysis of the Exendin-4-Loaded Nanoparticles.

All the samples were analyzed without any pretreatment. 30 μL of each sample was placed in an FTIR with an attenuated total reflectance (ATR) accessory. The spectra were collected with an average of 20 scans per sample from 500 to 4000 cm⁻¹. Before each measurement, a background correction was performed to avoid atmospheric interference and reduce instrumental noise.

Lyophilization of Exendin-4-Loaded Nanoparticles

Exendin-4-loaded nanoparticle suspensions were aliquoted into glass vials. The vials were immersed in liquid nitrogen for 10 min to snap-freeze the solutions and then lyophilized using an FreeZone Triad Benchtop Freeze Dryers (Labconco, USA) at 25° C. and 10 Pa for 36 h. The freeze-dried powder was stored at −80° C. The size and polydispersity index of the lyophilized nanoparticles were monitored upon reconstitution at each time point.

In Vitro Release Profiles of Exendin-4 from Nanoparticles

NP-2, NP-3, NP-4 and NP-5 suspensions (1 mL) were pipetted into dialysis tubes (MWCO 300 kDa) and incubated in 9 mL of PBS (10 mM, pH 7.4, with 0.05% w/v sodium azide) at 37° C. on a shaker (100 rpm). At predetermined time points (1, 2, 3, 4, 5, 6, 7, 8, 10, 12, 16 and 18-day), 200 μL of the sample was collected from the PBS incubation medium and was analyzed to determine the exendin-4 concentration using NanoOrange® Protein Quantitation Kit. After sampling, 200 μL fresh PBS was added to keep a constant volume of the incubation medium. To test the release profile at environments with different ionic strengths, a series of concentration of the PBS incubation medium (10, 20 and 30 mM) were used on NP-4 with the same experimental set-up and concentration of released exendin-4 examined at day 1, 2, 3, 4, 5 and 6.

Cell Viability Evaluation Upon Nanoparticle Dosage: MTT Assay

Caco-2 cells were cultured in tissue culture flasks using Dulbecco's modified Eagle's medium (DMEM) supplemented with high glucose, 10% fetal bovine serum (FBS), 1% nonessential amino acids, 1% L-glutamine, 1% penicillin-streptomycin (100 IU/mL) at 37° C. in a 5% CO2 incubator. For the cytotoxicity assay, Caco-2 cells were seeded into 96-well plates at a density of 1.0×10⁴ cells per well and allowed to grow for 24 h. The nanoparticle suspensions were added to the culture and incubated for 24 h. 20 μL of MTT assay solution (5 mg/mL) was added into each well and incubated for 4 h. Upon removal of the solution in each well, 150 μL of DMSO was added to dissolve the formazan crystals for 10 min and the amount of MTT-formazan was determined by absorbance at 570 nm.

Animal Study Protocols

All animal procedures were performed in accordance with an approved protocol by the Animal Care and Use Committee at Jiangsu Province Academy of Traditional Chinese Medicine, Nanjing, China. T2D db/db C57BL/KsJ (db/db) and normal C57BL/KsJ (db/m) and Balb/c mice were purchased from Model Animal Research Center of Nanjing University.

In Vivo Release Profiles of Exendin-4 from Nanoparticles

Exendin-4 was labeled by Cy 7.5 to track the in vivo release profile of exendin-4 following intraperitoneal (i.p.) administration. Nanoparticles were prepared using 1 mg/mL Cy 7.5 labeled exendin-4 (Cy7.5-exendin-4), 3 mg/mL TA and 0.05 mg/mL Fe³⁺ following the same procedures as described in the previous section. After i.p. injection of 200 μL of nanoparticle suspension or free exendin-4 (n=3 Balb/c mice per group, dose=500 μg/kg), the biodistribution of Cy 7.5-exendin-4 was revealed by near infrared imaging using the Perkin Elmer IVIS system (Hopkinton, USA) with ex 780 nm and em 810 nm at 1, 3, 6, 24, 48, 72, 96, 120, 144, 168, 192 and 216 h post-administration. Blood samples were collected from orbital venous plexus at each time point and the fluorescence intensity in blood was monitored.

Measurement of Blood Glucose Levels and Pharmacokinetic Analysis of Exendin-4 Loaded Nanoparticles.

The in vivo glycemic control was tested using 8-week male db/db mice, which are genetically obese leptin receptor-deficient mice typically used as a spontaneous T2D mouse model. Mice were randomly divided into four groups (n=5 model mice per group) and i.p. injected with PBS, free exendin-4 solution (3 mg/kg), low dose of NP-4 (3 mg/kg) and high dose of NP-4 (6 mg/kg), respectively. Healthy mice (db/m) were used as a control. Blood samples were collected from tail vein and blood glucose level was determined in duplicates using a blood glucose meter (OneTouch® UltraVue, Johnson, USA). The area under curve (AUC) of the blood glucose level versus time was calculated to evaluate the therapeutic effect.

The oral glucose tolerance test (OGTT) was conducted to test the glucose-stabilizing capability of exendin-4-loaded nanoparticles (NP-4). The db/db mice were randomly divided into three groups and fasted overnight (˜16 h). The control group was treated with PBS and the other groups were i.p. administrated with a single dose of NP-4 (1.5 mg/kg) or free exendin-4 (1.5 mg/kg). After 1 h, glucose in PBS was given via oral gavage at a dose of 2 g/kg. The blood glucose level was then monitored at 0, 1, 2, 3, 4 and 5 h using the blood glucose meter. The AUC of blood glucose level versus time in response to OGTT was calculated.

To study the pharmacokinetics, the db/db mice were randomly divided into two groups and dosed by single i.p. injection of free exendin-4 and NP-4 (1.5 mg/kg), respectively. Blood samples were collected from orbital venous plexus at predetermined time points (0, 1, 3, 6, 12, 24, 48, 72, 96, 120, 144, 168 and 192 h) followed by centrifugation at 4000 rpm for 20 min. Serum exendin-4 concentration was then quantified using ELISA kits. The AUC of serum exendin-4 concentration versus time was calculated.

Effect of Exendin-4-Loaded Nanoparticle Treatment on the Function and Pathology of Major Organs.

Blood samples were collected from orbital venous plexus and then centrifuged at 4000 rpm to separate the serum. The levels of AST, ALP, ALT, γ-GT, BUN and CR in blood were then determined. The liver, kidneys, spleen, lungs, and small intestine of the mice were harvested and fixed by 4% paraformaldehyde. The organs were sliced using paraffin sectioning method and dried. The slices were observed after standard H&E staining.

Statistical Analysis

All values are expressed as mean±S.D. Comparisons among all groups were evaluated using One-way ANOVA or t-test by Graph-Pad Prism version 5.0 for Windows (GraphPad Software, US), and p<0.05 was considered to be statistically significant.

### Example 1

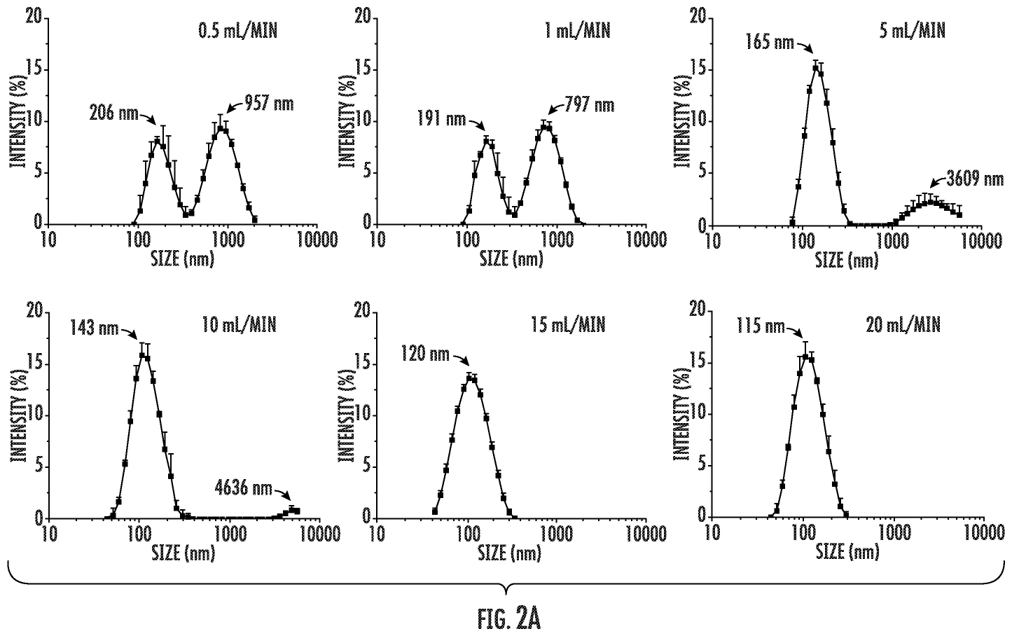

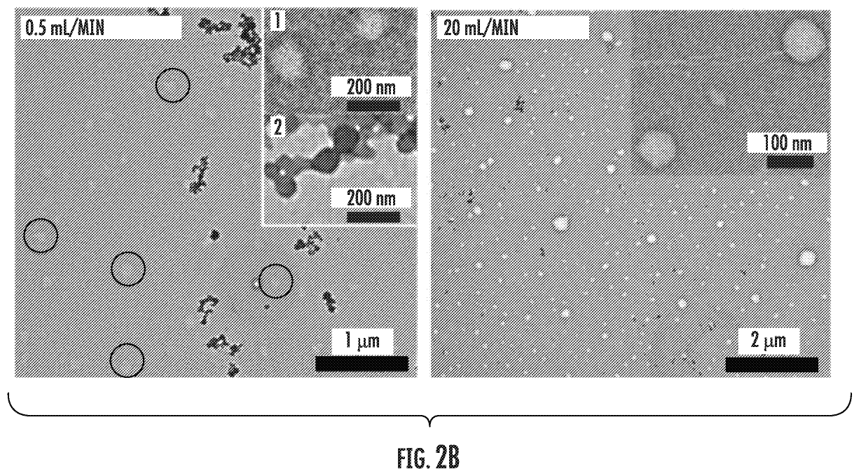

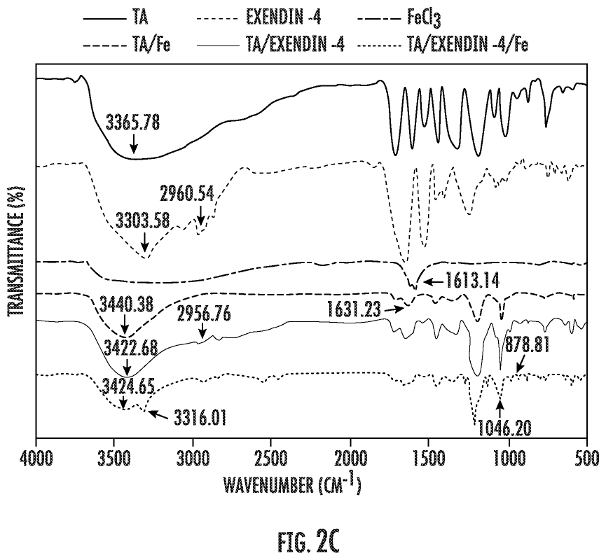

The rapid homogeneous mixing was achieved by a three-inlet CIJ device that has been widely used in FNC [26, 27, 30, 31] and flash nanoprecipitation (FNP) processes [32, 33] (FIG. 1). The inventors demonstrated that a higher mixing efficiency is associated with a higher volumetric flow rate by each jet that gives rise to turbulent mixing inside the microchamber [27, 28]. At a low flow rate of 0.5 mL/min, there were two species with peak sizes of 206 nm and 957 nm as revealed by DLS analysis (FIG. 2A) and confirmed by TEM observations (FIG. 2B), where small nanoparticles (within red circles, FIG. 2B-1) coexisted with large aggregations (FIG. 2B-2). The inventors attribute the aggregate formation to inefficient mixing, leading to over-growth of the particles and multiple species of particles. As the flow rate increased from 0.5 to 20 mL/min, the average size of nanoparticle species decreased from 206 nm to 115 nm (FIG. 2B); and the larger sized particle species disappeared (FIG. 2A). When increasing the flow rate beyond 20 mL/min, the nanoparticle size did not decrease further, whereas the PDI slightly increased (FIG. 9). It appeared that 20 mL/min was the optimal flow rate for the preparation of these ternary nanoparticles.

Successful encapsulation of exendin-4 into nanoparticles and the interactions among different components were assessed by FTIR (FIG. 2C). The stretching vibration peak of the free hydroxyl groups of TA (phenolic hydroxyl) at ˜3360 cm⁻¹ shifted to 3422 cm⁻¹, 3440 cm⁻¹ and 3424 cm⁻¹ after TA was mixed with exendin-4, Fe³⁺ or both, respectively. The results indicate hydrogen bond formation between TA and exendin-4, as well as chelation coordination between TA and Fe³⁺.

This FNC process for nanoparticle preparation is scalable (Table 1), as the samples collected at different output volumes showed identical nanoparticle size, PDI and surface charge.

TABLE 1
Consistency of FNC with different batch scales for
preparation of NP-4

| | Batch Size (mL) | | | | |
|---|---|---|---|---|---|
| | 5 | 20 | 50 | 100 | 200 |
| Average | 114.5 ± | 116.2 ± | 115.9 ± | 115.2 ± | 114.5 ± |
| Size (nm) | 1.7 | 1.4 | 2.3 | 2.1 | 2.7 |
| PDI | 0.17 ± | 0.16 ± | 0.14 ± | 0.15 ± | 0.14 ± |
| | 0.02 | 0.01 | 0.01 | 0.02 | 0.01 |

All data are shown as mean ± S.D. (n = 6).

The nanoparticles can further be lyophilized for long-term storage. Typically, it is necessary to include sufficient amount of cryoprotectants (e.g. glucose, trehalose, etc.) to maintain the stability of nanoparticles during freeze-drying process. Nonetheless, cryoprotectants were not required for the FNC-produced TA/exendin-4/Fe³⁺ nanoparticles, as they maintained the same size, PDI and surface charge profile after lyophilization of the original nanoparticle suspension in the absence of any cryoprotectant. These nanoparticles also preserved the same physical profile after reconstituting samples that had been in storage at −80° C. for 3 months (Table 2). This was presumably due to the hydrophilic nature of TA molecules on nanoparticle surfaces; more importantly, the abundant surface phenolic groups from the TA molecules could form hydrogen bonds with water molecules, thus effectively serving as a “self-cryoprotectant” during lyophilization and reconstitution.

TABLE 2
Characteristics of freshly prepared vs.
lyophilized-and-reconstituted NP-4 nanoparticles.

| Parameter | Freshly prepared | Reconstituted |
|---|---|---|
| Average size (nm) | 114.3 ± 1.8 | 112.5 ± 2.4 |
| Zeta potential (mV) | −22.5 ± 0.8 | −23.1 ± 0.5 |
| Polydispersity index (PDI) | 0.14 ± 0.01 | 0.15 ± 0.01 |
| Rehydration time (sec) | — | ~5 |

All data are shown as means ± S.D. (n = 6).

### Example 2

Effect of TA Concentration in FNC Preparation on Nanoparticle Characteristics and In Vitro Exendin-4 Release Profiles

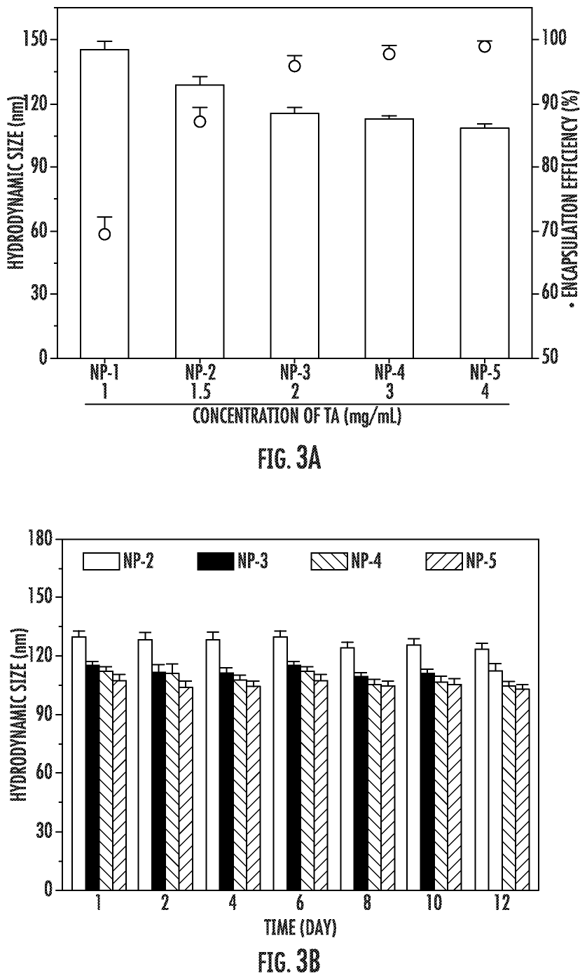

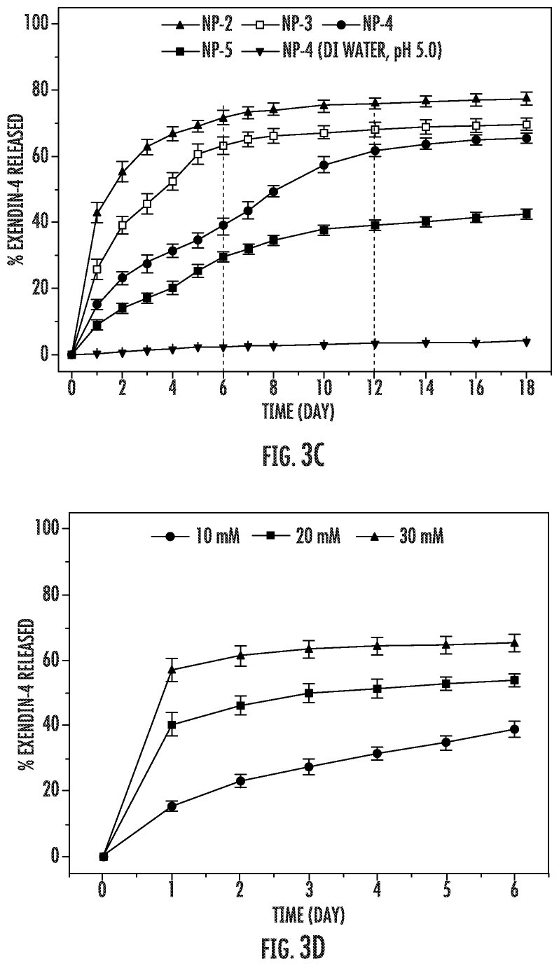

With an increased TA concentration from 1 to 4 mg/mL in FNC preparation, the average size of the ternary nanoparticles had decreased from 140 nm for NP-1 to 115 nm for NP-5. The encapsulation efficiency of exendin-4 increased from 60% to nearly 100% (FIG. 3A). As exendin-4 is encapsulated into nanoparticles through its interaction with TA, a higher TA concentration upon mixing provides higher loading capacity in nanoparticles. Besides, a higher TA concentration may generate a higher crosslinking density and a greater degree of compaction of the polypeptide exendin-4 upon complexation. It may also lead to a higher density of TA on nanoparticle surface, which generates a higher level of negative charges on the surface of growing nanoparticles, thus leading to early kinetic arrest due to charge repulsion and a smaller nanoparticle size. These nanoparticle formulations with different TA concentrations maintained high colloidal stability for at least 12 days without noticeable aggregates (FIG. 3B).

To understand the release mechanism of exendin-4 from nanoparticles, the inventors examined the effect of medium pH, ionic strength, and nanoparticle composition on the release profile of exendin-4. In deionized water at pH˜5, there was almost no release (˜2%) within 6 days (FIG. 3C). Since the pKa of tannic acid is around 6, at the weak acid condition (pH<6), the phenol groups in TA remain neutral form, which is critical to hydrogen bond formation between TA and exendin-4 and coordination complexation between TA and Fe³⁺ (FIG. 1B), thus retaining exendin-4 in the nanoparticles. As the pH increases to 7.4, ionization of the phenol groups will break down the hydrogen bond and coordination complexation crosslinks, leading to the dissociation of exendin-4 from the complexes in the nanoparticles (FIG. 1B).

Considering the lower encapsulation efficiency of exendin-4 in NP-1 (<60%), the in vitro release performance was compared only among NP-2, NP-3, NP-4 and NP-5 in PBS (10 mM, pH 7.4) at 37° C. As shown in FIG. 3C, NP-2 and NP-3 both had a significant burst release of exendin-4 (60% and 40%, respectively) in the first 2 days and quickly reached a plateau in approximately 6 days. When the TA concentration increased during formulation for NP-4 and NP-5, the burst release was reduced, and the overall release profile could be extended to 12 days. This can be attributed to increased crosslinking density with higher TA concentration in the nanoparticles that renders decreased dissociation rate of exendin-4 from nanoparticles.

The ionic strength can influence the release kinetics of exendin-4 (FIG. 3D). When the ionic strength was increased from 10 to 30 mM (PBS, pH 7.4), the burst release of exendin-4 became more appreciable with the first-day release going from 15% to 55%.

### Example 3

In Vivo Release Behavior Upon i.p. Injection of Exendin-4-Loaded Nanoparticles

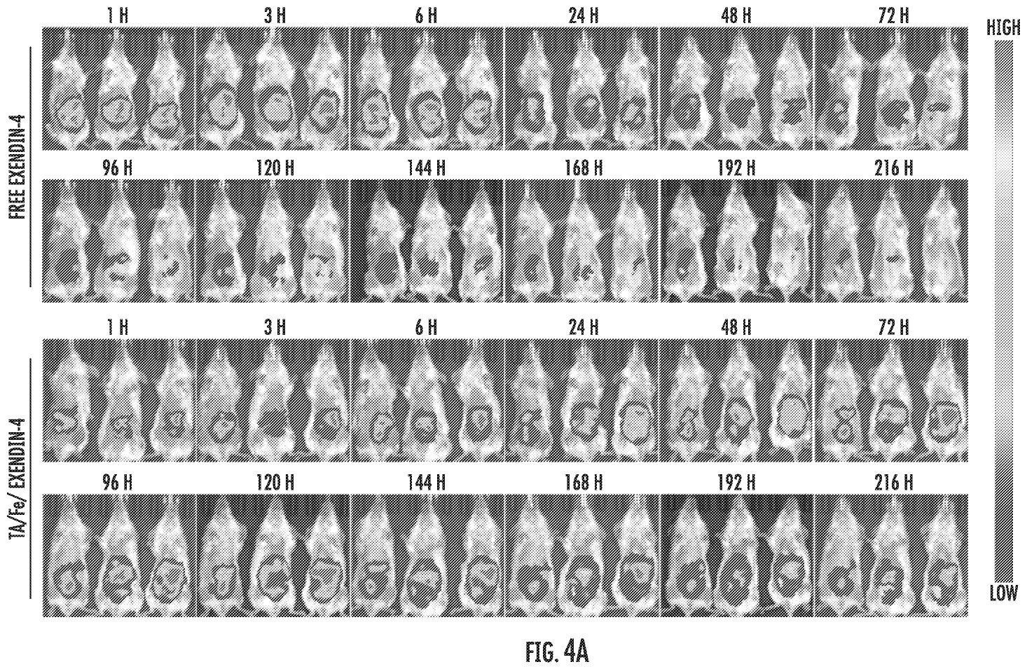

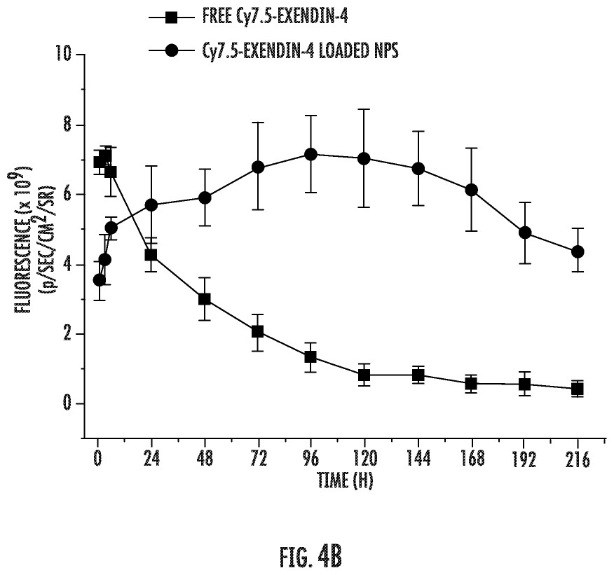

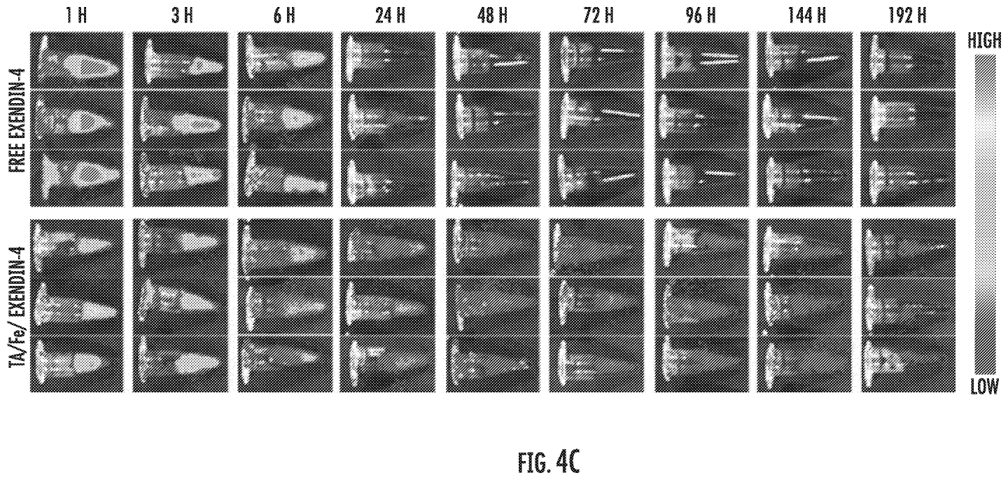

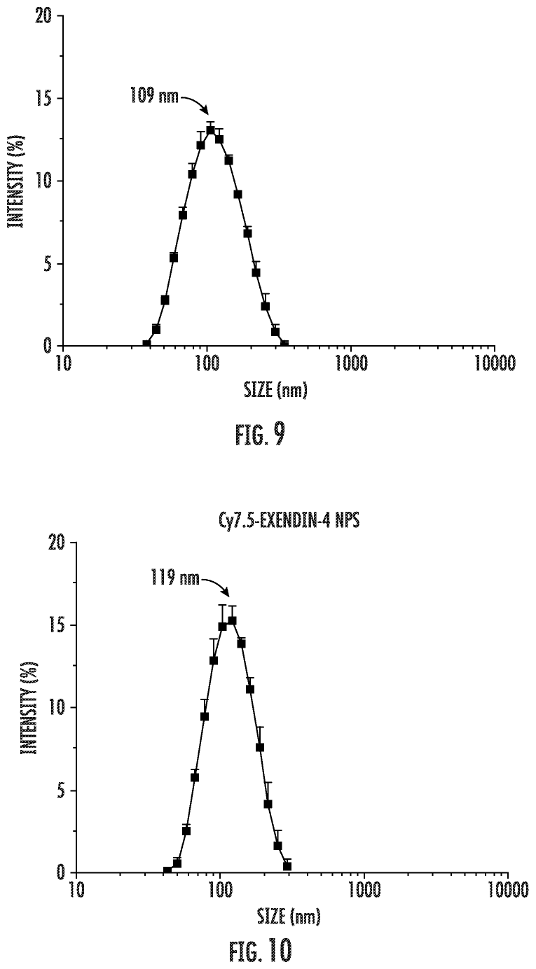

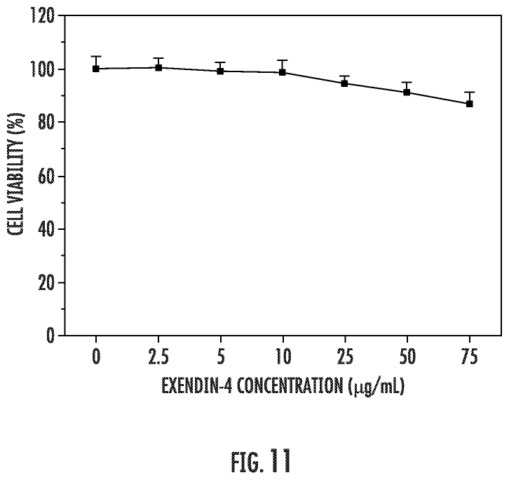

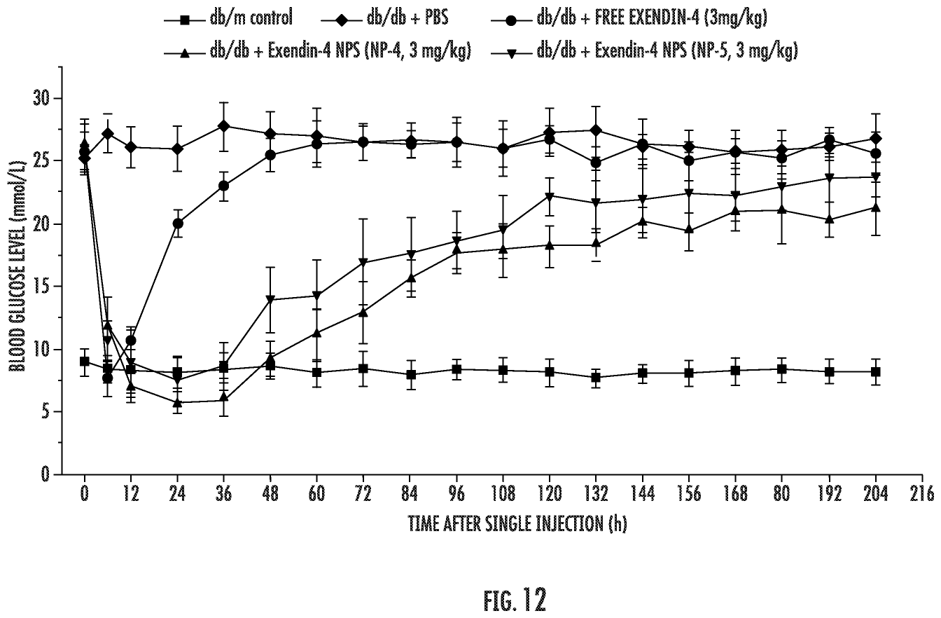

The Cy 7.5-labeled nanoparticles showed no significant difference in nanoparticle size compared with the unlabeled nanoparticles (FIG. 10). MTT assay was conducted to assess cytotoxicity and showed no significant decrease in cell viability when nanoparticles were incubated with Caco-2 cells for 24 h, within 0 to 75 μg/mL (FIG. 11). The results of fluorescence imaging of mice treated with different formulations were shown in FIG. 4A. Notably, with the same dose, the signal intensity of free exendin-4 group in the peritoneal cavity was much stronger than that of exendin-4 loaded nanoparticles group. This was due to fluorescence quenching within the nanoparticles and a shield effect by TA/Fe³⁺ complexes since their absorption range (400 to 1000 nm) covered the excitation and emission wavelengths of Cy 7.5 (ex: 780 nm, em: 810 nm) [20]. The signal of the free exendin-4 group dissipated gradually, and could hardly be detected after 48 h, due to quick diffusion and degradation of the peptide. In contrast, the signal intensity of the exendin-4-loaded nanoparticles group gradually increased and spread out within the peritoneal cavity as exendin-4 released from nanoparticles and escaped from fluorescence quenching and the shielding effect. The signal intensity peaked around 72 h and lasted until 144 h. This period correlated well with the linear release phase of this formulation (NP-4) in vitro. The signal gradually decreased after 144 h, as the release rate decreased gradually (FIG. 4A, B).

The exendin-4 blood concentration was assessed through fluorescence imaging of collected blood samples (FIG. 4C). Blood concentrations of exendin-4 from the free exendin-4 group peaked immediately after dosing and decreased quickly within 6 h. The peptide concentration reached a background level by 24 h, suggesting the fast clearance of the exendin-4 from injection site and the short half-life of exendin-4 in the blood [34]. Upon dosage of exendin-4-loaded nanoparticles, the detected blood drug concentrations were moderate within 3 h and maintained at a medium to low level for at least 192 h, matching the in vivo imaging results at the peritoneal cavity. Based on these findings, the exendin-4-loaded nanoparticles are capable to deliver exendin-4 for an extended period of time in vivo.

### Example 4

T2D Treatment with Exendin-4-Loaded Nanoparticles in Mouse Model

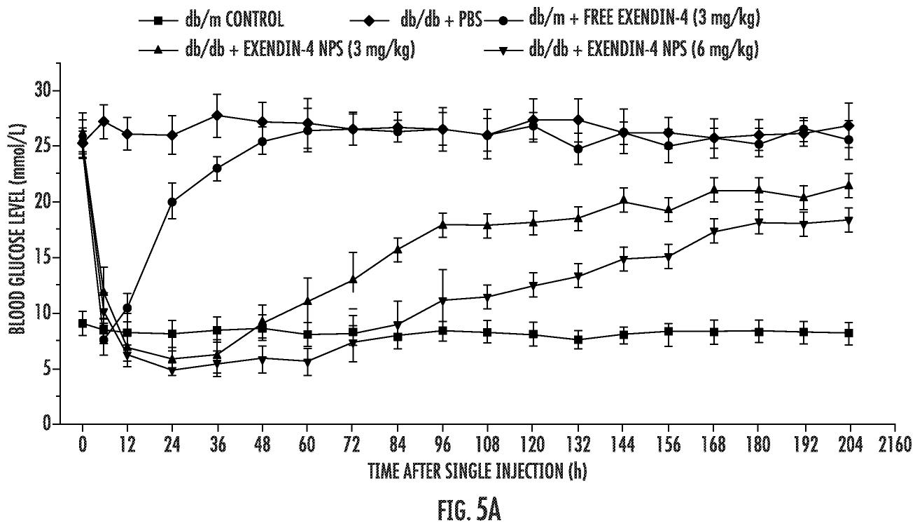

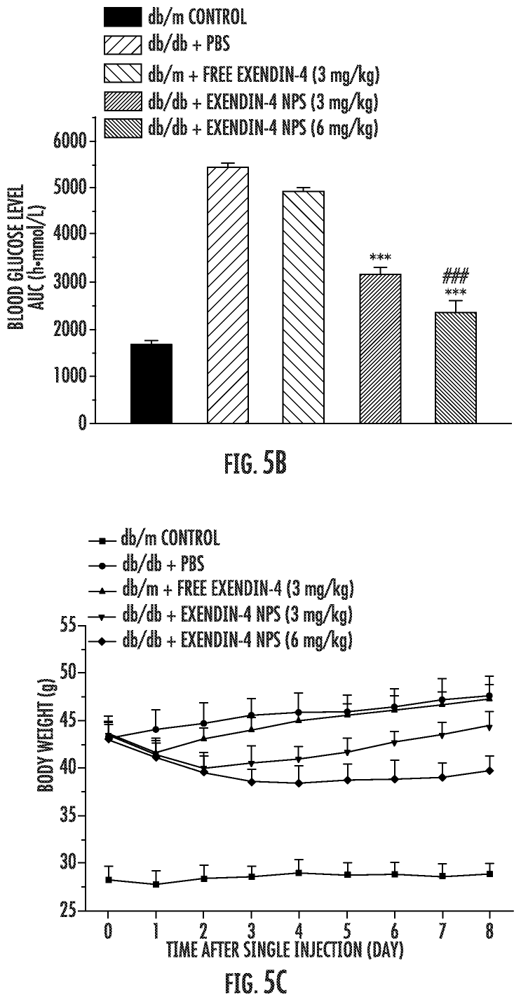

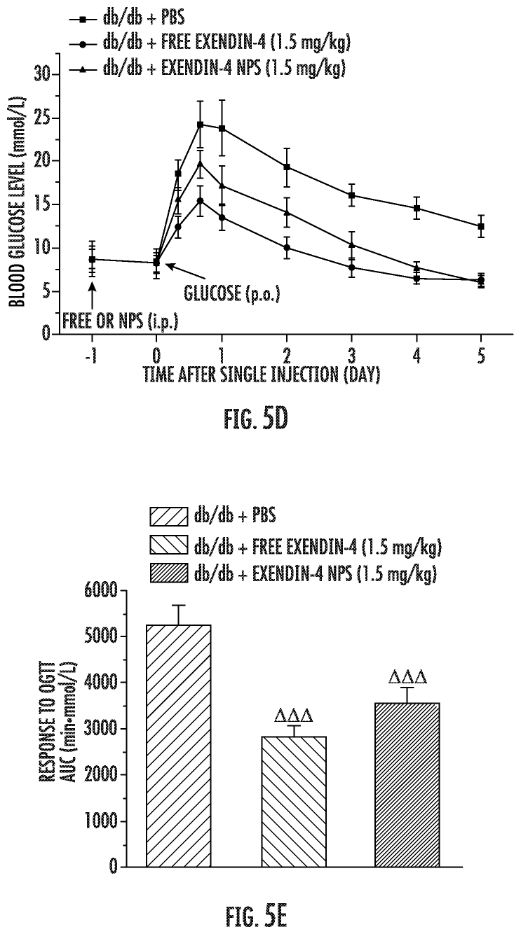

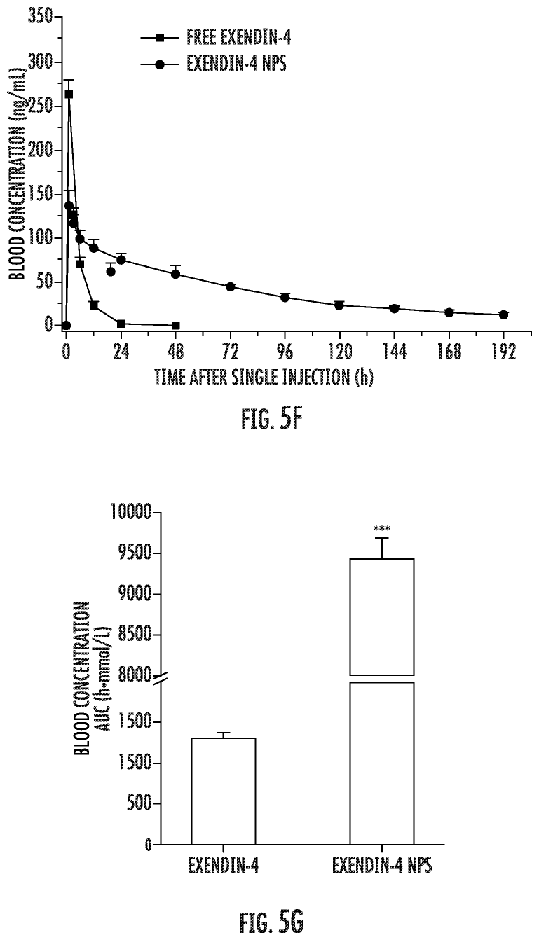

The effectiveness in controlling blood glucose level by each test group was shown in FIG. 5A. The healthy control group (db/m mice) and the model control group (db/db T2D mice) demonstrated the normal and high blood glucose level with low fluctuation throughout the experiments. When free exendin-4 was administrated to T2D mice, the blood glucose level was rapidly lowered to the level found in healthy mice within 6 h, but increased rapidly above an alert level (20 mmol/L) by 24 h and returned to the original level at 48 h post-administration. The lower dose NP-4 (3 mg/kg) administration also rapidly decreased the blood glucose level to the normal level within 12 h of dosing, and maintained that level for approximately 48 h with no obvious hypoglycemic symptoms. With a higher dosage of 6 mg/kg, the blood glucose reduction effect was similar to the lower dose group, but the normal blood glucose level was maintained for 72 h. Notably, the blood glucose level was still below 50% of the original level after 132 h, and gradually returned to 75% of the original level by 204 h, indicating its longer-acting regulatory effect of this nanoparticle formulation as a result of sustained release of exendin-4. The effect was also dose-dependent. The AUC (from 0 h to 204 h) was calculated to quantify the therapeutic effect (FIG. 5B), showing a significant improvement in blood glucose control from free exendin-4 group to high-dose NP-4 group. The low-dose NP-4 (3 mg/kg) and high-dose NP-4 (6 mg/kg) groups exhibited a trend of decreasing body weight compared to free exendin-4 group and T2D model control group (FIG. 5C), further demonstrating effective reduction of T2D symptoms. The release rate of exendin-4 have to be optimized to yield an exendin-4 concentration in the blood that is above the lower effective limit for successful treatment outcome. NP-5, which was formulated to have the slowest release rate, maintained the normal blood glucose level for slightly shorter duration (36 h) compared with NP-4 (48 h) when given at the same dose (3 mg/kg), and gave a faster relapse to hyperglycemic status (FIG. 12).

The oral glucose tolerance test (OGTT) was conducted to mimic a situation of pre-meal administration. As shown in FIGS. 5D and E, blood glucose level in the T2D model control group went through a sharp increase and peaked (˜24 mmol/L) after 40 min of glucose administration. With exendin-4 nanoparticles pre-dosed 1 h prior to glucose challenge, an appreciable reduction effect on blood glucose increase was observed with a peak blood glucose level of 17.0 mmol/L. In contrast, the effect of free exendin-4 injection was slightly higher with the peak blood glucose level of 13.5 mmol/L, as a result of faster absorption into circulation.

To further examine the long-term release profile in vivo, the inventors compared the pharmacokinetics of the exendin-4 given in free form and encapsulated in nanoparticles. The serum drug concentration in free peptide injection group peaked rapidly to 263 ng/mL within 1 h and declined to nearly zero at 24 h post injection (FIG. 5F). In contrast, the serum concentration of exendin-4 in the nanoparticle group was still detectable at 192 h after injection. The AUC of blood exendin-4 concentration profile of nanoparticle dosage was 7.2 times that of free exendin-4 injection. The inventors confirmed that NP-4 nanoparticle formulation qualifies for a long-acting system to treat T2D using exendin-4 with a lower dosing frequency and prolonged efficacy.

### Example 5

Pathological Improvement of Major Organs in T2D Mice Following Exendin-4 Nanoparticle Treatment

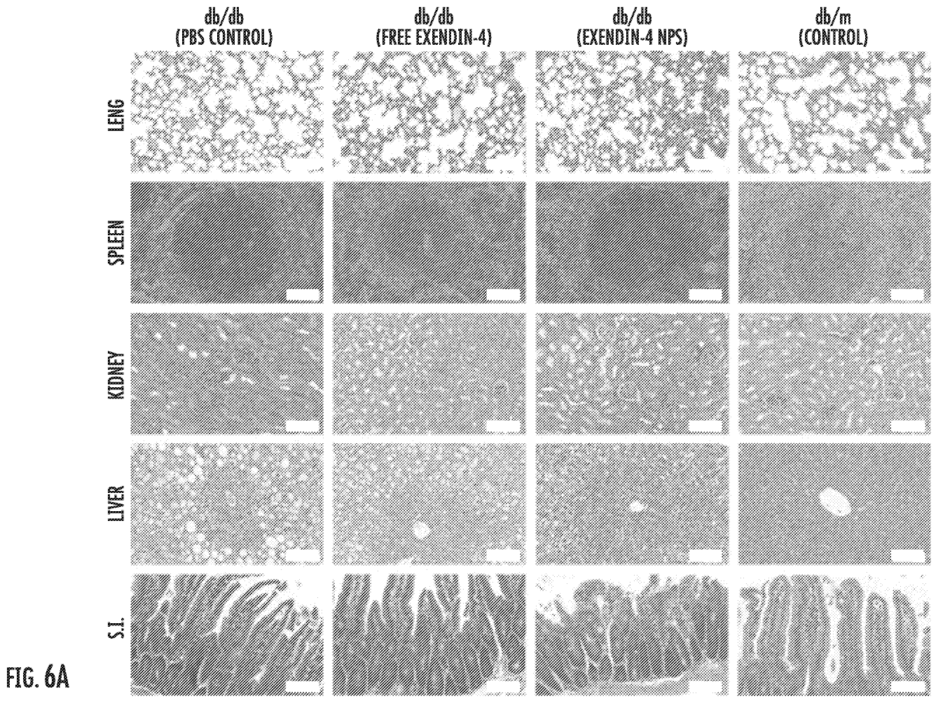

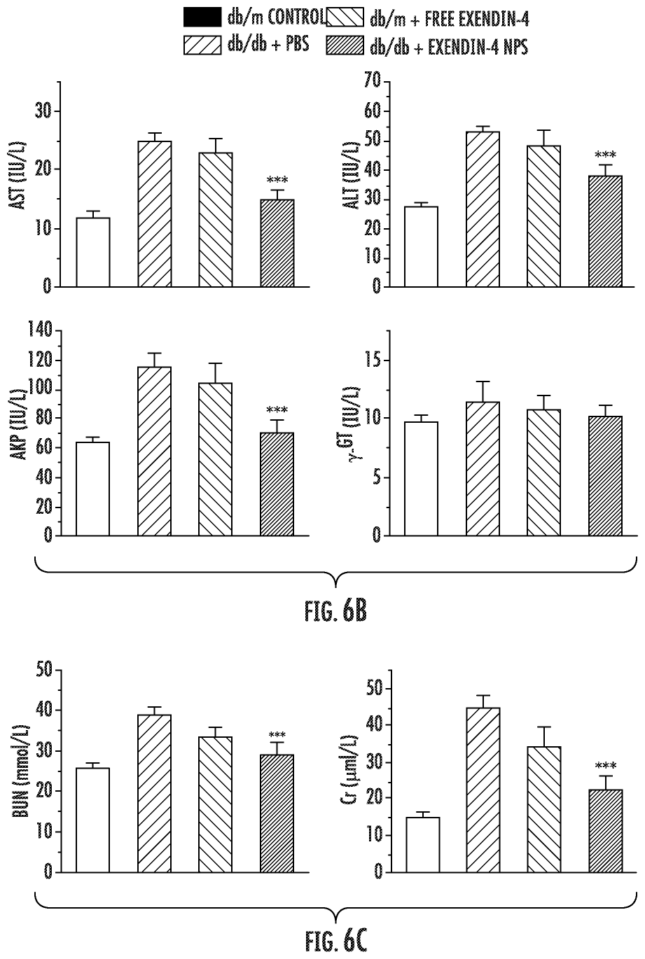

The health risks of T2D are manifested in tissue damage to major organs besides a high blood glucose level. The inventors further examined if the treatment by exendin-4-loaded nanoparticles could help relieve the relevant pathological damages in major organs. As shown in FIG. 6A, the hepatocytes were significantly swollen with regions of lytic necrosis found in the liver in the model control group. There was also hyperplasia in renal glomerulus and shrinkage in renal capsule, indicating a morbid condition in the kidney. At 9 days after administration of nanoparticles at a single dose of 3 mg/kg, the pathological damages to the liver and kidney were alleviated and the improvement was more pronounced than the free exendin-4 treatment. In addition, there was no obvious inflammation or any other pathological changes after NP-4 administration in all organs assessed.

Notably, the levels of ALP, ALT, AST, γ-GT, BUN and CR in T2D model control mice were all higher than those of healthy mice (FIG. 6B). After treatment of free exendin-4 or exendin-4-loaded nanoparticles, these pathological indices were all improved; and the overall improvement in the nanoparticle group was more significant. This was consistent with the histopathological assessment results. These observations indicated good biocompatibility and therapeutic effect of exendin-4-loaded nanoparticles in relieving T2D-associated symptoms beyond maintaining blood glucose level with a single treatment.

To summarize, the present inventors developed a TA/exendin-4/Fe³⁺ ternary nanoparticle system that is capable of releasing a water soluble drug, such as a peptide, in a sustained manner after intraperitoneal administration. Their use of the FNC technique facilitated a rapid mixing of the three components that enabled simultaneous complexation between TA and exendin-4, and TA/Fe³⁺ coordination to generate uniform and stable nanoparticles. Such a controlled complexation assembly is not possible without homogenous mixing afforded through FNC. The nanoparticles of the present invention exhibited excellent size uniformity and high encapsulation efficiency as a result of the efficient mixing using FNC techniques. These nanoparticles of the present invention can mediate a tunable release of drug (exendin-4), showed good biocompatibility, and can be stored in lyophilized form and reconstituted readily before use without the need of any cryoprotectant.

The in vivo study in the T2D mice showed that the optimized ternary nanoparticles effectively reduced blood glucose level rapidly and maintained glycemic control over several days following a single dose, and improved treatment outcomes in terms of liver and kidney damage and body weight control. It is important to note that FNC is a highly reproducible and scalable process for the production of complexation-mediated nanoparticle assembly for the encapsulation and delivery of macromolecule therapeutics [26, 27, 30, 31]. The carrier molecule TA used in this study is a naturally occurring food supplement found on the FDA GRAS (generally recognized as safe) list; and it is biocompatible when used in the appropriate concentration range [35]. Therefore, this nanoparticle system has a high potential of clinical translation as a long-acting T2D treatment nanomedicine.

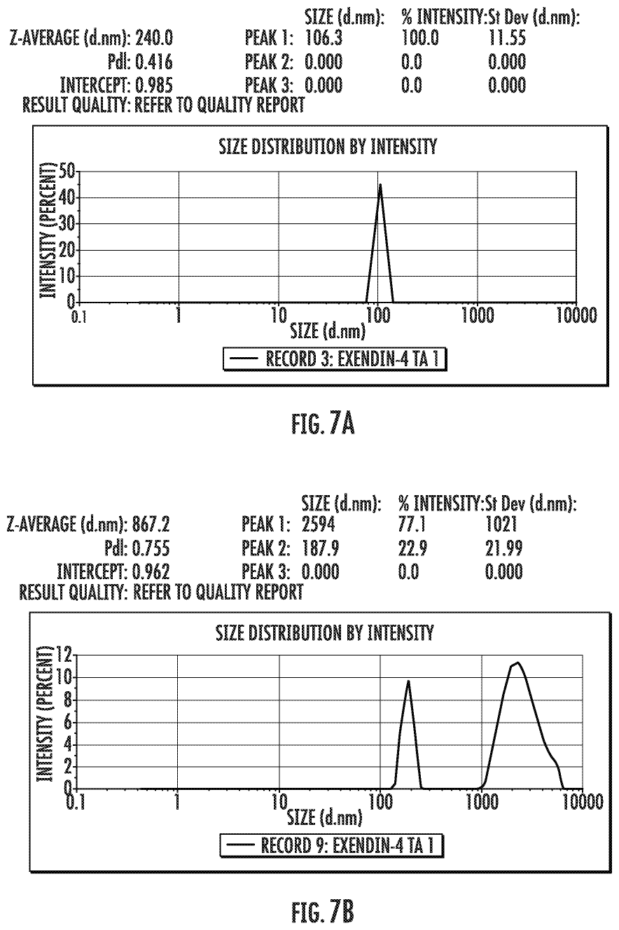

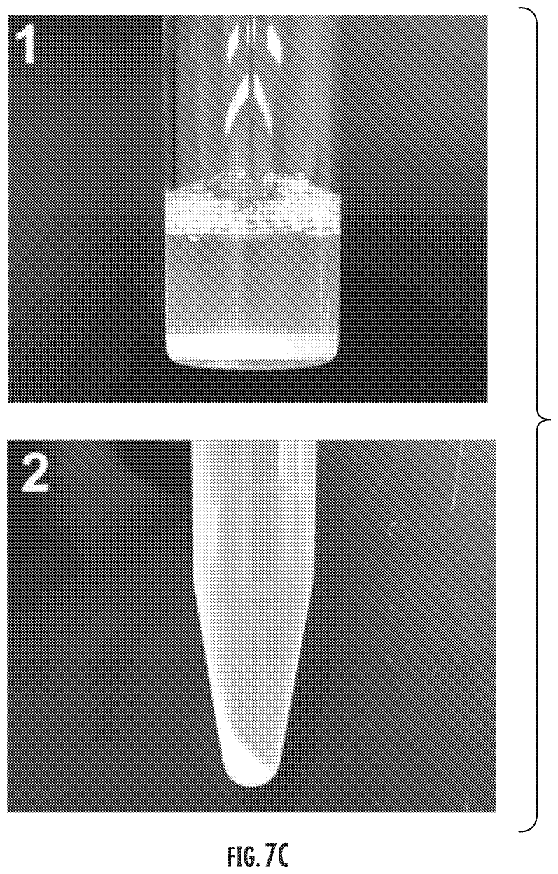

The inventors first tested the stability of TA/exendin-4 binary complexes by running FNC without Fe³⁺, and found that the complexes quickly aggregated within a few hours (FIG. 7A-7C).

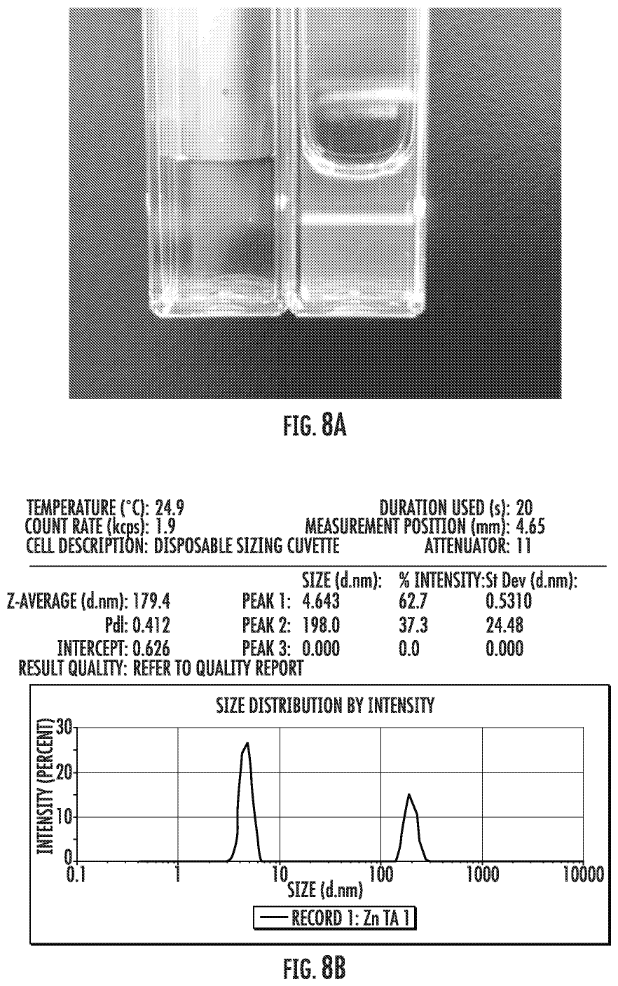

It was previously known that TA was mostly used to precipitate proteins [29]. The present inventors now show that a strong coordination interaction also exists between trivalent ion like Al³⁺ and tannic acid, which can serve to stabilize the TA/exendin-4 binary complexes (FIG. 8A-8B). However, preparations formulated with TA/exendin-4 with divalent ions such as Zn²⁺ did not yield detectable particles (FIG. 8A-8B). Stabilization of such complexes requires efficient quenching of the TA/exendin-4 complexation to freeze the nanoparticle structure and prevent their over-growth into larger aggregates. Here the inventors selected the coordination complexation between Fe³⁺ and TA to “saturate” the excess phenol groups and forming additional crosslinks within the nanoparticle structure as a stabilization mechanism (FIG. 1A). This approach also generates a negatively charged surface (zeta potential ˜23 mV), which creates charge repulsion to prevent aggregation following nanoparticle formation. Without being held to any particular theory, it is thought that in this system, TA binds with exendin-4 through hydrogen bonding and hydrophobic interactions, and with Fe³⁺ through phenol-Fe³¹ coordination for additional crosslinking and stabilization. As these complexation “reactions” occur at relatively high rates, the mixing kinetics of the 3 components needs to match the reaction rates in order to form uniform ternary complex nanoparticles, rather than TA-Fe³⁺ complexes or TA-exendin-4 complexes or heterogenous complex nanoparticles. Therefore, devising a process that allows the 3 components to mix homogeneously within milliseconds is a prerequisite for generating uniform nanoparticles.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

[1] M. Stumvoll, B. J. Goldstein, T. W. van Haeften, Type 2 diabetes: principles of pathogenesis and therapy, Lancet 365 (2005) 1333-1346.
[2] M. A. Nauck, J. J. Holst, B. Willms, W. Schmiegel, Glucagon-like peptide 1 (GLP-1) as a new therapeutic approach for Type 2-diabetes, Exp. Clin. Endocrinol. Diabetes. 105 (1997) 187-195.
[3] L. L. Baggio, D. J. Drucker, Biology of Incretins: GLP-1 and GIP, Gastroenterology 132 (2007) 2131-2157.
[4] G. Grunberger, A. Chang, G. Garcia Sodia, F. T. Botros, R. Bsharat, Z. Milicevic, Monotherapy with the once-weekly GLP-1 analogue dulaglutide for 12 weeks in patients with Type 2 diabetes: dose-dependent effects on glycaemic control in a randomized, double-blind, placebo-controlled study, Diabet. Med. 29 (2012) 1260-1267.
[5] M. A. Nauck, R. E. Ratner, C. Kapitza, R. Berria, M. Boldrin, R. Balena, Treatment with the human once-weekly GLP-1 analogue taspoglutide in combination with metformin improves glycemic control and lowers body weight in patients with type 2 diabetes mellitus inadequately controlled with metformin alone: a double-blind placebo-controlled study, Diabetes Care. 32 (2009) 1237-1243.
[6] M. N. Feinglos, M. F. Saad, F. X. Pi-Sunyer, B. An, O. Santiago, Effects of liraglutide (NN2211), a long-acting GLP-1 analogue, on glycaemic control and bodyweight in subjects with Type 2 diabetes, Diabet. Med. 22 (2005) 1016-1023.
[7] J. Eng, W. A. Kleinman, L. Singh, G. Singh, J. P. Raufman, Isolation and characterization of exendin-4, an exendin-3 analogue, from Heloderma suspectum venom. Further evidence for an exendin receptor on dispersed acini from guinea pig pancreas, J. Biol. Chem. 267 (1992) 7402-7405.
[8] D. Kim, L. MacConell, D. Zhuang, P. A. Kothare, M. Trautmann, M. Fineman, K. Taylor, Effects of Once-Weekly Dosing of a Long-Acting Release Formulation of Exenatide on Glucose Control and Body Weight in Subjects With Type 2 Diabetes, Diabetes Care 30 (2007) 1487-1493.
[9] L. L. Nielsen, A. A. Young, D. G. Parkes, Pharmacology of exenatide (synthetic exendin-4): a potential therapeutic for improved glycemic control of type 2 diabetes, Regul. Pept. 117 (2004) 77-88.
[10] H. Kim, J. Lee, T. H. Kim, E. S. Lee, K. T. Oh, D. H. Lee, E.-S. Park, Y. H. Bae, K. C. Lee, Y. S. Youn, Albumin-Coated Porous Hollow Poly(Lactic-co-Glycolic Acid) Microparticles Bound with Palmityl-Acylated Exendin-4 as a Long-Acting Inhalation Delivery System for the Treatment of Diabetes, Pharma. Res. 28 (2011) 2008-2019.
[11] H. Kim, H. Park, J. Lee, T. H. Kim, E. S. Lee, K. T. Oh, K. C. Lee, Y. S. Youn, Highly porous large poly(lactic-co-glycolic acid) microspheres adsorbed with palmityl-acylated exendin-4 as a long-acting inhalation system for treating diabetes, Biomaterials 32 (2011) 1685-1693.
[12] H.-H. Kwak, W.-S. Shim, M.-K. Son, Y.-J. Kim, T.-H. Kim, H.-J. Youn, S.-H. Kang, C.-K. Shim, Efficacy of a new sustained-release microsphere formulation of exenatide, DA-3091, in Zucker diabetic fatty (ZDF) rats, Euro. J. Pharm. Sci. 40 (2010) 103-109.
[13] C. Lee, J. S. Choi, I. Kim, H. J. Byeon, T. H. Kim, K. T. Oh, E. S. Lee, K. C. Lee, Y. S. Youn, Decanoic acid-modified glycol chitosan hydrogels containing tightly adsorbed palmityl-acylated exendin-4 as a long-acting sustained-release anti-diabetic system, Acta Biomater. 10 (2014) 812-820.
[14] C. Lee, J. S. Choi, I. Kim, K. T. Oh, E. S. Lee, E.-S. Park, K. C. Lee, Y. S. Youn, Long-acting inhalable chitosan-coated poly(lactic-co-glycolic acid) nanoparticles containing hydrophobically modified exendin-4 for treating type 2 diabetes, Int. J. Nanomedicine. 8 (2013) 2975-2983.
[15] J. Lee, C. Lee, T. H. Kim, E. S. Lee, B. S. Shin, S.-C. Chi, E.-S. Park, K. C. Lee, Y. S. Youn, Self-assembled glycol chitosan nanogels containing palmityl-acylated exendin-4 peptide as a long-acting anti-diabetic inhalation system, J. Control. Release, 161 (2012) 728-734.
[16] C. Wei, G. Wang, B. C. Yung, G. Liu, Z. Qian, and X. Chen, Long-acting release formulation of exendin-4 based on biomimetic mineralization for type 2 diabetes therapy, ACS nano 11 (2017) 5062-5069.
[17] E. Bakheet and F. Kratz, Impact of albumin on drug delivery-new applications on the horizon, J. Control. Release, 157 (2012) 4-28.
[18] C. Q. Pan, J. M. Buxton, S. L. Yung, I. Tom, L. Yang, H. X. Chen, M. MacDougall, A. Bell, T. H. Claus, K. B. Clairmont, J. P. Whelan, Design of a long acting peptide functioning as both a glucagon-like peptide-1 receptor agonist and a glucagon receptor antagonist, J. Biol. Chem. 281 (2006) 12506-12515.
[19] H. Chen, G. Wang, L. Lang, O. Jacobson, D. O. Kiesewetter, Y. Liu, Y. Ma, X. Zhang, H. Wu, L. Zhu, G. Niu, X. Chen, Chemical Conjugation of Evans Blue Derivative: A Strategy to Develop Long-Acting Therapeutics through Albumin Binding, Theranostics 6 (2016) 243-253.
[20] I. Phiwchai, W. Yuensook, N. Sawaengsiriphon, S. Krungchanuchat, C. Pilapong, Tannic acid (TA): A molecular tool for chelating and imaging labile iron, Euro. J. Pharm. Sci. 114 (2018) 64-73.
[21] J. Iglesias, E. Garcia de Saldaña, J. A. Jaén, On the Tannic Acid Interaction with Metallic Iron, Hyperfine Interact. 134 (2001) 109-114.
[22] P. K. South, D. D. Miller, Iron binding by tannic acid: Effects of selected ligands, Food Chem. 63 (1998) 167-172.
[23] L. Xie, R. L. Wehling, O. Ciftci, Y. Zhang, Formation of complexes between tannic acid with bovine serum albumin, egg ovalbumin and bovine beta-lactoglobulin, Food. Res. Int. 102 (2017) 195-202.
[24] A. M. Vissers, A. E. Blok, A. H. Westphal, W. H. Hendriks, H. Gruppen, J.-P. Vincken, Resolubilization of Protein from Water-Insoluble Phlorotannin-Protein Complexes upon Acidification, J. Agric. Food. Chem. 65 (2017) 9595-9602.
[25] J. P. Vanburen, W. B. Robinson, Formation of Complexes between Protein and Tannic Acid, J. Agric. Food. Chem. 17 (1969) 772-777.
[26] J. L. Santos, Y. Ren, J. Vandermark, M. M. Archang, J.-M. Williford, H.-W. Liu, J. Lee, T.-H. Wang, H.-Q. Mao, Continuous Production of Discrete Plasmid DNA-Polycation Nanoparticles Using Flash Nanocomplexation, Small 12 (2016) 6214-6222.
[27] Z. He, J. L. Santos, H. Tian, H. Huang, Y. Hu, L. Liu, K. W. Leong, Y. Chen, H.-Q. Mao, Scalable fabrication of size-controlled chitosan nanoparticles for oral delivery of insulin, Biomaterials 130 (2017) 28-41.
[28] B. K. Johnson, R. K. Prud'homme, Chemical processing and micromixing in confined impinging jets, AIChE. J. 49 (2003) 2264-2282.
[29] H. P. S. Makkar, K. Becker, Behaviour of tannic acid from various commercial sources towards redox, metal complexing and protein precipitation assays of tannins, J. Sci. Food Agric. 62 (1993) 295-299.
[30] Z. He, Z. Liu, H. Tian, Y. Hu, L. Liu, K. W. Leong, H.-Q. Mao, Y. Chen, Scalable production of core-shell nanoparticles by flash nanocomplexation to enhance mucosal transport for oral delivery of insulin, Nanoscale 10 (2018) 3307-3319.
[31] Z. He, Y. Hu, T. Nie, H. Tang, J. Zhu, K. Chen, L. Liu, K. W. Leong, Y. Chen, H.-Q. Mao, Size-controlled lipid nanoparticle production using turbulent mixing to enhance oral DNA delivery, Acta Biomater. 81 (2018) 195-207.
[32] W. S. Saad, R. K. Prud'homme, Principles of nanoparticle formation by flash nanoprecipitation, Nano Today. 11 (2016) 212-227.
[33] R. F. Pagels, J. Edelstein, C. Tang, R. K. Prud'homme, Controlling and Predicting Nanoparticle Formation by Block Copolymer Directed Rapid Precipitations, Nano Lett. 18 (2018) 1139-1144.
[34] D. Parkes, C. Jodka, P. Smith, S. Nayak, L. Rinehart, R. Gingerich, K. Chen, A. Young, Pharmacokinetic actions of exendin-4 in the rat: Comparison with glucagon-like peptide-1, Drug Dev. Res. 53 (2001) 260-267.
[35] K. T. Chung, Z. Lu, M. W. Chou, Mechanism of inhibition of tannic acid and related compounds on the growth of intestinal bacteria, Food Chem. Toxicol. 36 (1998) 1053-1060.

1. A nanoparticle composition comprising at least one tannic acid, at least one trivalent metal ion, and at least one biologically active agent.

2. The nanoparticle composition of claim 1, wherein said biologically active agent comprises one or more water-soluble proteins, water-soluble peptides, water soluble small molecules, or a combination thereof.

3. The nanoparticle composition of either of claim 1 or 2, wherein the mass ratio of the tannic acid to the trivalent metal ion in the range between 10 and 100.

4. The nanoparticle composition of any of claims 1 to 3, wherein the trivalent metal ion is selected from the group consisting of Fe (III), Al (III) or a combination thereof.

5. The nanoparticle composition of any of claims 1 to 4, wherein the biologically active agent is a peptide comprising 5 to 150 amino acid residues.

6. The nanoparticle composition of any of claims 1 to 4, wherein the biologically active agent is a protein, wherein the molecular mass of the protein have a size in the range from 5 to 200 kDa.

7. The nanoparticle composition of any of claims 1 to 6, wherein the biologically active agent is selected from the group consisting of an antibody; an antibody fragment; a hormone; a hormone receptor; a receptor ligand; a cytokine; a growth factor; or a combination thereof.

8. The nanoparticle composition of any of claims 1 to 7, wherein the nanoparticle comprises 30-60% (w/w) of the tannic acid, 0.1-20% (w/w) of the trivalent metal ion, and 1-50% (w/w) of the biologically active agent.

9. The nanoparticle composition of any of claims 1 to 8, wherein the biologically active agent is exendin-4.

10. The nanoparticle composition of claim 9, wherein said composition comprises 65.6% (w/w) of tannic acid, 32.8% (w/w) of exendin-4, and 1.6% (w/w) of trivalent metal ion Fe (III).

11. The nanoparticle composition of claim 9, wherein said composition comprises 79.2% (w/w) of tannic acid, 19.8% (w/w) of exendin-4, and 1.0% (w/w) of trivalent metal ion Fe (III).

12. Use of the nanoparticle compositions of any of claims 1-11 for treatment of a disease in a subject.

13. Use of the nanoparticle compositions of claim 9, for treatment of Type 2 Diabetes in a subject in need thereof.

14. A flash nanocomplexation (FNC) method of continuously generating uniform tannic acid/peptide or protein/trivalent metal ions nanoparticles, comprising:
(a) flowing a first stream comprising tannic acid dissolved in water having a concentration of the tannic acid in the range of 0.2 to 40 mg/mL at a first variable flow rate into a confined chamber;
(a) flowing a second stream comprising one or more of a water-soluble peptide or water-soluble protein having a concentration in the range of 0.1 to 20 mg/mL at a second variable flow rate into the confined chamber;
(b) flowing a third stream comprising one or more of a water-soluble trivalent metal ion having a concentration in the range of more 0.005 to 1 mg/mL at a third variable flow rate into the confined chamber; and
(c) impinging the first stream, the second stream and the third stream in the confined chamber, thereby causing the tannic acid, the one or more water-soluble trivalent metal ions and the one or more water-soluble peptide or water-soluble protein to undergo a complexation process that continuously generates tannic acid/peptide or protein/metal ions (III) ternary nanoparticles.

15. The method of claim 14 wherein the confined chamber is a 3-inlet confined impingement jet mixer device.

16. The method of claim 14 wherein the first, the second, and the third variable flow rate are each in the range from 0.5 to 100 mL/min.

17. The method of claim 14, wherein the one or more water-soluble trivalent metal ions is selected from the group consisting of Fe (III), Al (III), or a combination thereof.

18. The method of claim 14 wherein the tannic acid concentration is in the range from 0.5 to 10 mg/ml and the first stream has a pH in the range of 3.0 to 7.0.

19. The method of claim 14 wherein the water-soluble peptide or protein concentration is in the range from 0.5 to 5 mg/ml and the second stream has a pH in the range of 5.5 to 8.0.

20. The method of claim 14 wherein the water-soluble trivalent metal ion concentration is in the range from 0.005 to 0.5 mg/ml and the third stream has a pH in the range of 1.0 to 4.0.

21. The method of claim 14, wherein the generated tannic acid/peptide or protein/metal ions (III) ternary nanoparticles have a size in the range from about 20 nm to about 500 nm in diameter.

22. The method of claim 14, wherein the generated tannic acid/peptide or protein/metal ions (III) ternary nanoparticles have a polydispersity index in the range from about 0.05 to about 0.3.


SEQUENCE LISTING (3 sequences)

SEQ ID NO: 1 - protein, 39 aa, Heloderma suspectum
HGEGTFTSDLSKQMEEEAVRLFIEWLKNGGPSSGAPPPS

SEQ ID NO: 2 - protein, 31 aa, Artificial Sequence
  synthetic sequence
HAEGTFTSDVSSYLEGQAAKEFIAWLVRGRG

SEQ ID NO: 3 - protein, 44 aa, Artificial Sequence
  synthetic sequence
HGEGTFTSDLSKQMEEEAVRLFIEWLKNGGPSSGAPPSKKKKKK